(19) United States
(12) Patent Application Publication
(10) Pub. No.: US 2003/0113761 A1
(43) Pub. Date: Jun. 19, 2003

(54) Modelling of biochemical pathways

(76) Inventors: Patrick Tan, Singapore (SG); Kumar Selvarajoo, Singapore (SG)

Correspondence Address: DANN DORFMAN HERRELL & SKILLMAN, SUITE 720, 1601 MARKET STREET, PHILADELPHIA, PA, 19103-2307, US

(21) Appl. No.: 10/222,029

(22) Filed: Aug. 16, 2002

Related U.S. Application Data
(60) Provisional application No. 60/312,938, filed on Aug. 16, 2001.

(51) Int. Cl. C12Q 1/68; G01N 33/53; G06F 19/00; G01N 33/48; G01N 33/50

(52) U.S. Cl. 435/6; 435/7.1; 702/20

(57) ABSTRACT

A method of investigating a biochemical pathway, especially a biochemical pathway treated with a test substance which is being considered as a potential drug, in order to predict its effects, comprises the steps of:

storing, e.g. in computer memory a set of mathematical expressions which are effective to calculate values representing pre-selected metabolite concentrations in a reference biochemical pathway from an initial value representing the measured concentration of one metabolite in that reference biochemical pathway;

assaying the concentration of one or more metabolites in one or more test samples of a second biochemical pathway containing the same metabolites as the reference pathway, and storing values representing the measured concentrations;

using said mathematical expressions to calculate a predicted value of concentration of at least one metabolite in the second biochemical pathway from an initial value representing the measured concentration of one metabolite in the second pathway;

comparing such predicted values with corresponding values representing measured metabolite concentrations to obtain a comparison relationship; and deriving a quality measure of the match between two biochemical pathways on the basis of the comparison relationship.

Also included in the invention are apparatus for carrying out the method and a computer memory product carrying at least one digital file which is a program to carry out the method.

### FIELD OF THE INVENTION

The present invention relates to the mathematical modelling of biochemical pathways, which may be linear or branched pathways, cycles or networks.

### BACKGROUND OF THE INVENTION

It has long been known that biochemical transformations of one molecule into another frequently proceed through a considerable number of intermediate metabolites with each transformation being catalysed by an enzyme. Such systems may incorporate regulatory mechanisms such as positive or negative feed-back mechanisms or feed-forward mechanisms. There is an emerging recognition that in vivo, metabolic systems can display properties such as robustness, or ultrasensitivity, or oscillatory behaviour [3].

Mathematical models of biochemical pathways, cycles or networks, are potentially valuable for understanding these systems. A potential advantage of such a mathematical model is that it may lead to significant reductions in the time and cost associated with traditional biological research.

Several computational approaches are currently being used to model complex systems. These include kinetic methodologies such as Michaelis-Menten (M-M) kinetics and metabolic control analysis (MCA), as well as stoichiometric approaches such as flux balance analysis (FBA) [4,5].

Traditionally, one daunting challenge facing the kinetic approaches has been that all kinetic parameters used in the model have to be known before the model can even be constructed [6]. Stoichiometric methodologies such as FBA do not require detailed kinetic information for model construction, and instead rely upon mass-action constraints to mathematically represent the biologic ‘state’ of a system [5]. Since FBA relies upon the creation of a pre-specified ‘closed’ metabolic network, any model tends to be confined to the metabolic system for which it was created. [7]. Thus, it is evident that there is a critical need for the development of novel modeling techniques which are not subject to the same limitations.

One common feature of both the kinetic and stoichiometric approaches is that they rely upon a ‘bottom-up’ approach, in which the first step in the modelling processes is to break down a complex system into minimal constituent units (usually enzymes). Mathematical relationships are then defined between these units in an attempt to replicate higher-level system behaviour. One inherent limitation in such methods, however, is that predictive accuracy can be difficult to obtain, as errors introduced at lower levels (e.g. via experimental measurements) can collectively accumulate as the model progresses through higher levels of complexity. In addition, for the kinetic approaches, most of the parameters have been determined using in vitro conditions, where the enzyme in question has been deliberately purified from its physiologic neighbours [8]. Thus, it remains an open question if these readings accurately reflect the actual behaviour of these enzymes in vivo.

### SUMMARY OF THE INVENTION

In one aspect the present invention provides a method of creating a representation of a biochemical pathway comprising a first metabolite and a plurality of further metabolites whose concentrations are directly or indirectly dependent on the concentration of the first metabolite, by steps of

choosing a set of mathematical expressions for calculating predicted values representing concentrations of said further metabolites from an initial value representing a measured concentration of the first metabolite in an embodiment of the biochemical pathway;

modifying some or all of the expressions so as to match the calculated values to a set of reference values representing measured concentrations in the embodiment of the biochemical pathway; and

storing the expressions in memory, for example on a data carrier such as computer-readable tape or disc, or in memory of a computer.

The mathematical model which is created may serve for use in research, diagnosis, identification of drug targets, drug discovery, drug screening, drug design, or evaluation of the mode of action, side effects or toxicity of a drug or other biologically active material.

The method may also comprise assaying one or more samples of said pathway to determine the initial and reference values representing measured concentrations.

Suitably the initial and reference values are mean values from measurement in a plurality of different samples all embodying the same biochemical pathway but taken from different members of the same species.

This method desirably includes a further step of using these expressions to calculate predicted values representing concentrations of said further metabolites from a value representing the concentration of a metabolite (which may be the first metabolite) measured in a second embodiment of the biochemical pathway and comparing these predicted values with values representing measured concentrations in the second embodiment of the pathway, in order to validate the set of expressions as a model for the biochemical pathway. This second embodiment of the biochemical pathway may occur in the same species as the first embodiment, but at a different site.

This methodology is a ‘top-down’ approach to modelling a biochemical pathway. In this approach, direct empirical relationships are constructed between individual products and substrates, and intervening catalytic enzymes are treated as ‘black boxes’. In this strategy, detailed enzyme kinetic information is not required for model construction, although complex regulatory events such as allosteric effects can still be incorporated. Known bottom-up approaches are heavily reliant on deduction from enzyme kinetic information in order to construct the model, and are vulnerable to an accumulation of errors arising from uncertainty in the original information. By contrast, the top-down approach utilised by this invention constructs relationships between individual products and substrates on an empirical basis, formulating a set of mathematical expressions to constitute a model and then adjusting that model to fit available data. Existing knowledge of what happens in the biological pathway may be used when selecting the form of mathematical expressions but adjustment of the model to fit available data does not require detailed kinetic information, as in ‘bottom-up’ approaches. This method uses fewer parameters than the known ‘bottom-up’ approaches which helps to minimise the chance of error accumulation from parameter uncertainty. The term pathway is used here to include linear or branching pathways, cyclic pathways or networks. Biochemical pathways include metabolic pathways in which there is interaction of compounds in a cascade or pathway involving the use of an enzyme to catalyze the reaction of one compound into another—these will include cycles (glycolysis, gluconeogenesis, krebs, carbon, nitrogen fixing, urea, photosynthesis etc). However, biochemical pathways also include cascading reactions, e.g. ligand/receptor interaction and signaling, for example tyrosine kinase reactions within a cell, immuno-complement cascade, lipid/carbohydrate/protein breakdown and synthesis etc.

In another aspect the present invention provides a method of investigating a biochemical pathway comprising:

storing a set of values representing measured concentrations of a set of metabolites in a biochemical pathway under investigation;

calculating predicted values of concentrations of one or more metabolites in said pathway from an initial value representing the measured concentration of one other metabolite in said pathway under investigation;

comparing one or more said predicted values with corresponding values representing the measured concentrations of the metabolites to obtain a comparison relationship; and

deriving a quality measure of the match between the biochemical pathway under investigation and another biochemical pathway containing these metabolites on the basis of the comparison relationship.

Storing of the values may be storage in some form of memory or data carrier, for example in computer memory or on computer-readable tape or disc.

This method may include a step of assaying or otherwise measuring concentrations of metabolites in at least one test sample embodying the biochemical pathway under investigation prior to storing values representing those concentrations.

Calculation of predicted values is preferably carried out using a stored set of mathematical expressions which model the relationship of concentrations of metabolites observable in the other biochemical pathway, which may be a known biochemical pathway constituting a reference. The method therefore preferably comprises providing, for example providing on a computer-readable data carrier, and/or storing in memory, a set of mathematical expressions which are effective to calculate values representing metabolite concentrations in a reference biochemical pathway, and the calculation of predicted values is performed using these mathematical expressions.

So, in a further aspect, the present invention provides an investigative method comprising:

storing a set of mathematical expressions which are effective to calculate values representing pre-selected metabolite concentrations in a pre-selected, reference biochemical pathway from an initial value representing the measured concentration of one metabolite in said reference biochemical pathway

storing a set of values representing measured concentrations of the metabolites in a second biochemical pathway containing the same metabolites as the reference pathway;

using said mathematical expressions to calculate a predicted value of concentration of one or more (preferably a plurality of) said metabolites in a subject biochemical pathway containing the same metabolites as the reference pathway from an initial value representing the measured concentration of one metabolite in said subject pathway;

comparing one or more said predicted values with corresponding values representing measured metabolite concentration to obtain a comparison relationship; and

deriving a quality measure of the match between two biochemical pathways on the basis of the comparison relationship. These two pathways may be the reference pathway and the subject pathway.

In significant forms of this invention, the subject pathway is also the second pathway, so that such forms of the invention provide an investigative method comprising:

storing a set of mathematical expressions which are effective to calculate values representing pre-selected concentrations of metabolites in a pre-selected, reference biochemical pathway from an initial value representing the measured concentration of one metabolite in said reference biochemical pathway

storing a set of values representing measured concentrations of the metabolites in a second biochemical pathway which is under investigation, containing the same metabolites as the reference pathway;

using said mathematical expressions to calculate a predicted value of concentration of one or more (preferably a plurality of) said metabolites in the second biochemical pathway which is under investigation from an initial value representing the measured concentration of one metabolite in said second pathway;

comparing one or more said predicted values with the corresponding values representing measured metabolite concentrations to obtain a comparison relationship; and

deriving a quality measure of the match between the second biochemical pathway which is under investigation and the pre-selected, reference biochemical pathway on the basis of the comparison relationship.

Once again, the methods of the invention may include a step of assaying or otherwise measuring concentrations of metabolites in at least one test sample embodying a biochemical pathway prior to storing values representing those concentrations. It is of course possible that the number of metabolites whose concentrations are actually measured will not be the same as the number for which predictions of concentration could be calculated using the mathematical expressions.

Storing of the mathematical expressions and likewise storage of the values representing the measured concentrations may be storage in some form of memory or data carrier, notably computer memory or computer-readable data carrier. The mathematical expressions may be incorporated within a stored program for a computer.

These methods preferably include an additional step of calculating further predicted values of the concentrations of some of said metabolites in a biochemical pathway under investigation from another initial value representing the measured concentration of another one of the metabolites in said pathway (i.e. a metabolite other than the metabolite represented by the first-mentioned initial value);

then comparing the further predicted values with the values representing measured concentrations so as to obtain a further comparison relationship; and

deriving a further quality measure of the match between biochemical pathways on the basis of the further comparison relationship.

This additional step may be carried out repeatedly, calculating predicted values from successive initial values representing the measured concentrations of different individual metabolites in the biochemical pathway under investigation. Such repetition can be useful for locating the point at which the biochemical pathway under investigation differs from the reference biochemical pathway.

The invention may be utilised to examine differences between a known biochemical pathway which serves as a reference and another biochemical pathway suspected to differ from the reference pathway even though the same metabolites occur in it. However, once a mathematical model has been created for a reference biochemical pathway, it can be used to investigate an embodiment of the same pathway in which there is a suspected abnormality, possibly of genetic origin, or an embodiment of the pathway which has been disturbed by exposure to a drug or other substance.

Hence, this method of investigating a biochemical pathway may constitute a method of detecting or locating abnormalities in the biochemical pathway under investigation.

It may constitute a method of diagnosis of disease states associated with abnormality in a biochemical pathway. In such circumstances, the known, normal form of the same pathway would be the reference pathway.

The method may constitute a method of screening for drugs effective in modifying a biochemical pathway or a method of testing the effect of compounds (e.g. drugs, pathogens) on a biochemical pathway.

For example, it may be desirable to determine the effects of a substance on a particular biochemical pathway. This may be to screen for potential drugs capable of modifying the pathway or for determining the negative side effects of known drugs or substances on the pathway in the hope of using this information to develop a counteracting substance. Thus methods according to this invention may also comprises treating a biochemical pathway with a test substance in order to provide the biochemical pathway under investigation.

A method of investigating the effect of a drug or other substance on a biochemical pathway may comprise the following steps:

storing a set of mathematical expressions which are effective to calculate values representing pre-selected concentrations of metabolites in a pre-selected, reference biochemical pathway from an initial value representing the measured concentration of one metabolite in said reference biochemical pathway;

treating an embodiment of a biochemical pathway with one or more test substances and thereafter assaying a test sample to measure concentrations of a plurality of metabolites lying within the treated biochemical pathway;

storing values representing said measured concentrations;

using said mathematical expressions to calculate a predicted value of concentration of one or more metabolites in the treated pathway under investigation from a value representing the measured concentration of one other metabolite in said pathway;

comparing one or more said predicted values with the corresponding values representing the measured concentrations of the metabolites to obtain a comparison relationship; and

deriving a quality measure of the match between the biochemical pathway treated with the test substance(s) and the pre-selected, reference biochemical pathway

A notable characteristic of these forms of the invention is that the predicted values are calculated from an initial value which represents one measured concentration value in the pathway under investigation and compared with other measured concentration values of the same pathway, after which the quality of match in that comparison provides information about similarity between that pathway and another, reference, pathway

In accordance with the present invention, a mathematical model can be created for a reference pathway and applied to a pathway under examination. As will be illustrated below, the inventors have exemplified the invention using a glycolytic reaction pathway. However, it will be apparent to the skilled person that the invention may be applied to various biochemical pathways which may themselves be linear or branched, cyclic or networked.

After creating a mathematical model for a reference pathway in accordance with this invention, it is possible to use it to predict concentrations in more than one pathway containing the same metabolites and to use it to make comparison between different pathways or even to make comparison between predicted values in one pathway and values representing measured concentration in another. So, the above-mentioned methods which use a model for a reference pathway to provide predictions for a second pathway containing the same metabolites can be extended by performing analogous steps for a third pathway containing the same metabolites. This third pathway may be a pathway which has been treated with a test substance. Such a method of the invention may include optimizing the set of mathematical expressions for the second pathway before applying the resulting model to the third pathway.

Thus, once a model has been created it is then possible, by taking the concentration of a first metabolite, to predict a set of metabolite concentrations in a sample comprising said first metabolite and a plurality of further metabolites as in the reference pathway.

This prediction may be considered as a control.

In order to test the effects of one or more substances on the biochemical pathway, the sample or a comparable sample can then be contacted with said one or more test substances to create a third version of the pathway.

Following treatment with the substance(s), the measured concentration of a first metabolite may be taken and, using the mathematical model already produced, a set of predicted metabolite concentrations in this version of the pathway) may be created. This step is preferably repeated at least once, more preferably 3 or 4 times, using the measured concentration values of the second, third, fourth etc, metabolites until a plurality of sets of predicted metabolite concentrations have been created. These may be considered as test predictions.

The test predictions are then compared with the control predictions. Divergence between the control and the test predictions will indicate an effect of the (one or more) substances on the biochemical pathway. By creating a plurality of test predictions, it is possible to determine at which point, in relation to the metabolites, the substance(s) made an effect on the pathway.

This knowledge will allow screening for drugs capable of correcting an abnormality in a biochemical pathway. This abnormality may be as a result of a disease state or as a consequence of an administered drug. Further, this knowledge may be used to check that potential drugs do not have side effects causing abnormalities to biochemical pathways.

The method of this invention may also be utilized as a method of determining, by detecting or identifying, differences in biochemical pathways between species (human v bacterial pathogen etc) to assess possible medical treatments.

An overall investigative method, in which a computational model is created and used can be stated as an investigative method for a biochemical pathway comprising a first metabolite and a plurality of further metabolites whose concentrations are directly or indirectly dependent on the concentration of the first metabolite, comprising steps of:

choosing a set of mathematical expressions for calculating predicted values representing concentrations of said further metabolites from an initial value representing a measured concentration of the first metabolite in a reference biochemical pathway;

modifying at least some of the expressions so as to match the calculated values to a set of reference values representing measured concentrations in the reference biochemical pathway; and

storing the expressions in memory;

storing a set of values representing measured concentrations of the metabolites in a second biochemical pathway containing the same metabolites as the reference pathway;

using said mathematical expressions to calculate predicted values of concentration of a plurality of said metabolites in the second pathway from an initial value representing the measured concentration of one metabolite in said second pathway;

comparing predicated values with the corresponding values representing measured metabolite concentrations in said second pathway to obtain a comparison relationship; and

deriving a quality measure of the match between said second biochemical pathway and the pre-selected, reference biochemical pathway on the basis of the comparison relationship.

The methods of this invention are likely to be computerised by carrying out the steps of calculation and comparison using a general purpose computer running a program devised to implement the calculations and comparisons required by one or more of the above methods.

This invention thus includes a computer program, which when run on a general purpose computer will carry out one or more of the above methods, and also includes a computer program, when recorded on a data carrier, for carrying out any of the above methods. This may be stated as a computer memory product having stored thereon at least one digital file, said memory product comprising computer readable memory and said stored digital file or files constituting a program to carry out one or more of the above methods.

Such a program can be expected to include code representing a set of mathematical expressions which are effective to calculate values representing pre-selected concentrations of metabolites in a pre-selected, reference biochemical pathway from an initial value representing the measured concentration of one metabolite in said reference biochemical pathway. Alternatively, instead of including code which represents the required set of mathematical expressions, the program may incorporate code which is effective, when the program is run on a data processing pathway, to receive input of and store said mathematical expressions or to receive input of and store selection of a set from among a stored collection of mathematical expressions.

Such a program can also be expected to include code which is effective when the program is run,

to receive input of and store values representing measured concentrations of metabolites in a biochemical pathway;

to perform the steps of using said mathematical expressions to calculate predicted values of concentrations from a value representing one measured concentration; comparing one or more said predicted values with corresponding values representing measured concentrations to obtain a comparison relationship; and deriving a quality measure of match on the basis of that comparison relationship; and

to display or output said quality measure.

Possibilities for display or output include graphical or numeric display, output to memory and output to a printer.

More specifically, in one aspect, the present invention provides a computer program for investigating a biochemical pathway comprising

(a) code representing a set of mathematical expressions which are effective to calculate values representing pre-selected concentrations of metabolites in a pre-selected, reference biochemical pathway from an initial value representing the measured concentration of one metabolite in said reference biochemical pathway; or

(b) code effective, when the program is run on a data processing system, to perform steps selected from: receive input of and store mathematical expressions, receive input and store selection from among a collection of stored expressions, and combinations of the two; plus

(c) code adapted to perform the following steps when the program is run on a data processing system:

receive input of and store a set of values representing measured metabolite concentrations in a biochemical pathway;

use said mathematical expressions to calculate a predicted value of concentration of one or more (preferably a plurality of) said metabolites in a pathway from an initial value representing the measured concentration of one metabolite in said pathway;

compare one or more said predicted values with corresponding values representing the measured concentrations of the metabolites, to obtain a comparison relationship;

derive a quality measure of the match between two biochemical pathways (notably between a pathway under investigation and the pre-selected, reference biochemical pathway) on the basis of the comparison relationship; and

display or output said quality measure.

The invention includes a computer memory product having any computer program as set forth above stored thereon at least one digital file.

In yet another aspect this invention includes apparatus for carrying out any of the above methods, and including

an input device for input of values representing concentrations of metabolites;

a processor for utilising a stored program to perform the steps of using said mathematical expressions to calculate predicted values of concentrations from a value representing one measured concentration, comparing one or more said predicted values with corresponding values representing measured concentrations to obtain a comparison relationship and deriving a quality measure of match on the basis of that comparison relationship;

a display device for said quality measure; and

memory for storage of values, said program and said quality measure.

An input device may comprise an assay device for measurement of concentrations of metabolites in a test sample.

Furthermore the invention includes a data carrier (which may be a computer memory product) having recorded thereon a mathematical model obtained by the method of the first aspect of this invention, and/or reference values as defined above, and/or a set of mathematical expressions as defined above.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1. (a) Schematic of the canonical human glycolytic reaction pathway, adapted from [14]. The glycolytic enzymes convert intracellular glucose into pyruvate through the series of reactions as depicted. The circled arrows indicate activation and the circled slash bars indicate inhibition. Under anaerobic conditions, pyruvate is converted to lactate while in aerobic conditions pyruvate enters the Krebs cycle. The abbreviations used are:

GLU, glucose; G6P, glucose-6-phosphate;

F6P, fructose-6-phosphate;

FBP, fructose-1,6-bisphosphate;

G3P, glyceraldehyde-3-phosphate;

DHAP, dihydroxyacetone phosphate;

BPG or 1,3-BPG, 1,3-bisphosphoglycerate;

3PG, 3-phosphoglycerate; 2PG, 2-phosphoglycerate;

PEP, phosphoenol-pyruvate;

ATP, adenosine triphosphate; ADP, adenosine diphosphate.

(b) Comparison of measured vs. predicted steady-state glycolytic metabolite concentrations for erythrocytes. The x-axis represents the various metabolites in the glycolytic pathway (G6P-PYR). The measured values are shown in full lines and the predicted values are in dashed lines. Both sets of values are also listed in Table 1.

FIG. 2. Comparison of measured vs. predicted steady-state glycolytic metabolite concentrations for myocytes. The measured concentration of BPG for myocytes is not known. The maximum deviation is observed at PEP. Note that at this point of maximum deviation the measured is obtained from a different source (Table 2).

FIG. 3. (a) The glycolytic pathway of T. brucei under aerobic conditions, adapted from [14]. T. brucei does not possess a functional Krebs cycle. The NADH generated during glycolysis is reoxidised by molecular oxygen using a dihydroxyacetone phosphate (DHAP): glycerol-3-phosphate (Gy3P) shuttle in combination with a terminal Gy3P oxidase in the mitochondrion. The abbreviations used are:

MIT, mitochondrion;

NAD+, nicotinamide adenine dinucleotide;

NADH, nicotinamide adenine dinucleotide, reduced;

Gy3P, glycerol-3-phosphate.

(b) Comparison of measured vs. predicted steady-state glycolytic metabolite concentrations for T. brucei (aerobic) using the HEGM. Measured values reflect trypasonomal glycolysis in an aerobic state. For the first 2 steps (G6P-F6P), the maximum deviation is at step F6P. Subsequent to F6P, the maximum deviation is at BPG.

(c) and (d) The HEGM was instructed to adopt measured concentrations of G3P(c), and BPG(d) respectively. Note the improvement in the predicted result in (d) compared with (c).

(e) Comparison of measured vs. predicted steady-state glycolytic metabolite concentrations for T. brucei (aerobic) using HEGM^{tr}. HEGM^{tr} is the original HEGM in which the steady-state coefficient describing the G3P to BPG transition has been altered. Note the improvement in the predicted result, compared with (b).

(f) Comparison of measured (solid line) vs. predicted (dotted line) steady state glycolytic metabolite concentrations for T. brucei (anaerobic) using TBAE. All metabolites are accurately predicted to within a factor of 2×, except for Gy3P where the deviation is 8.9×.

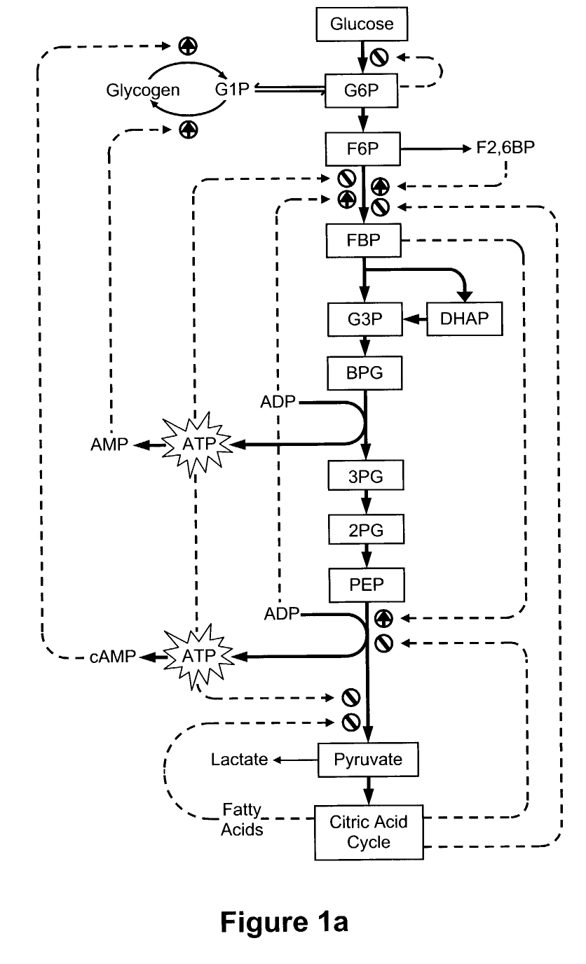

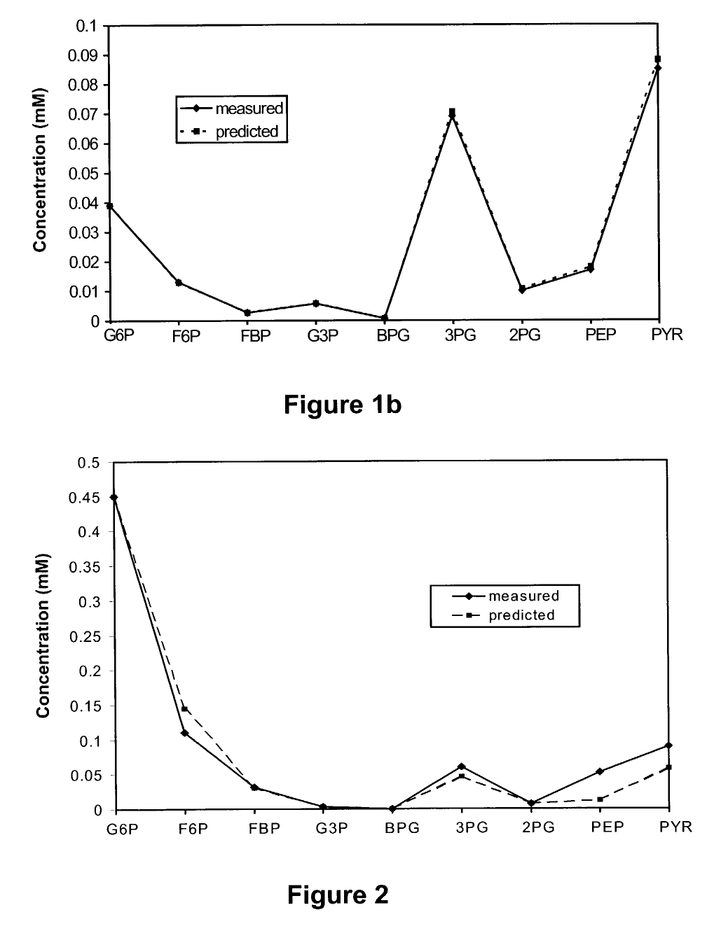

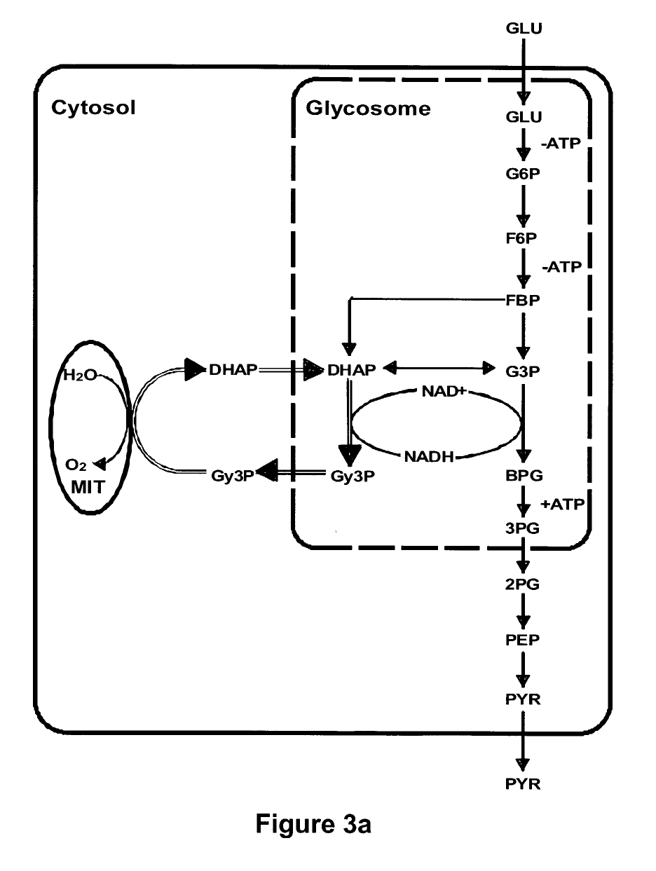

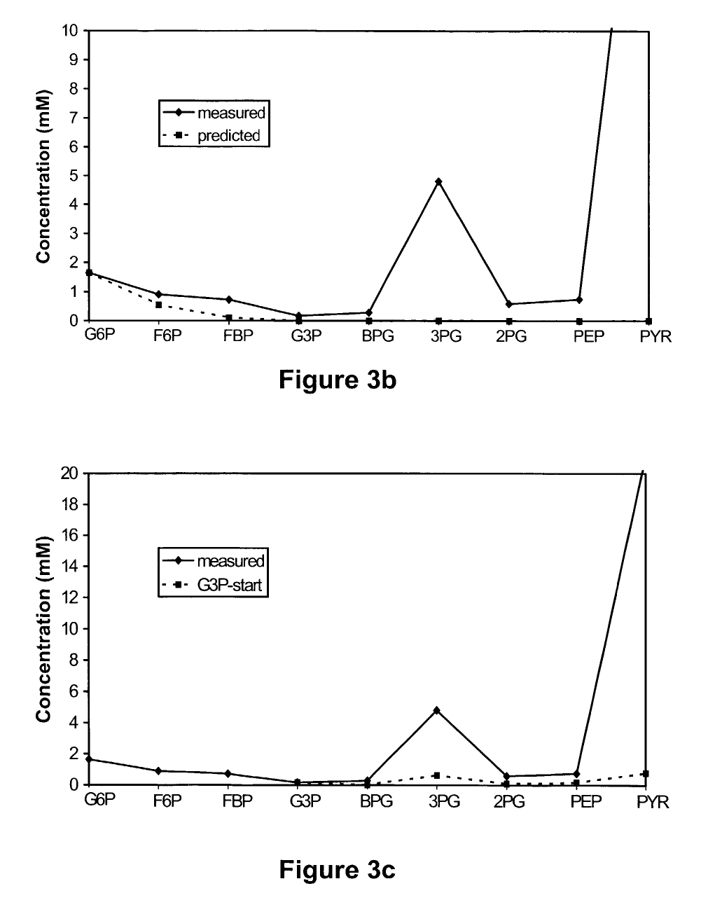

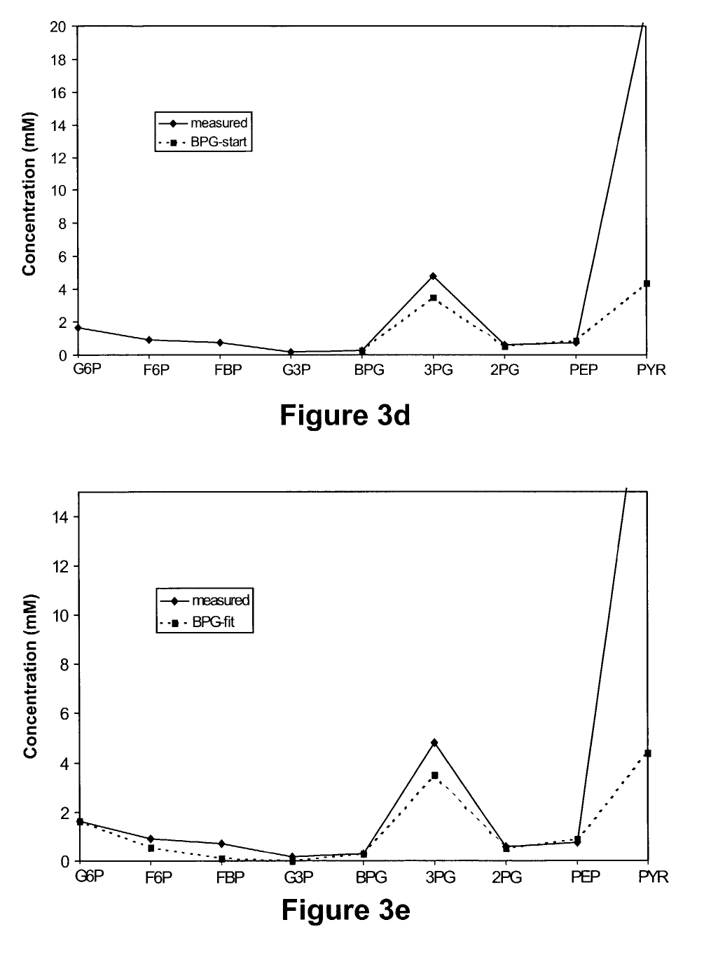

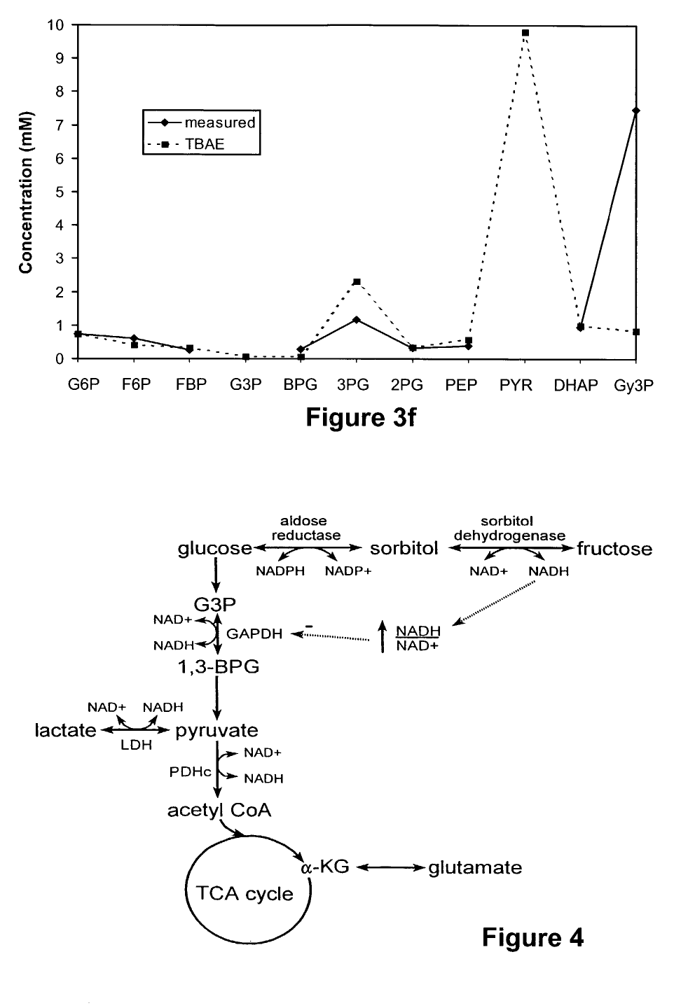

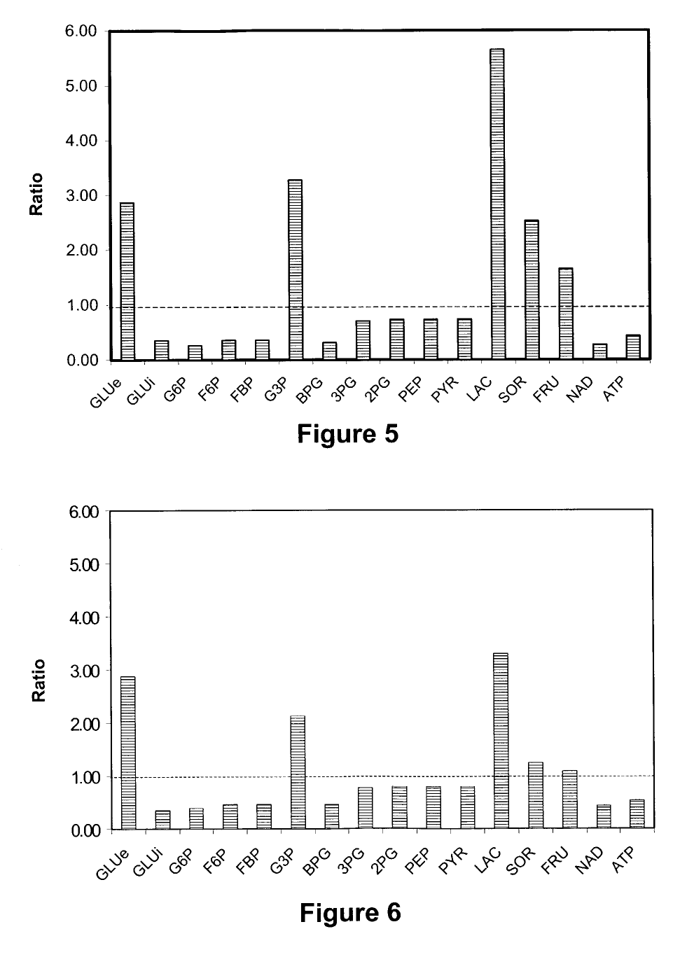

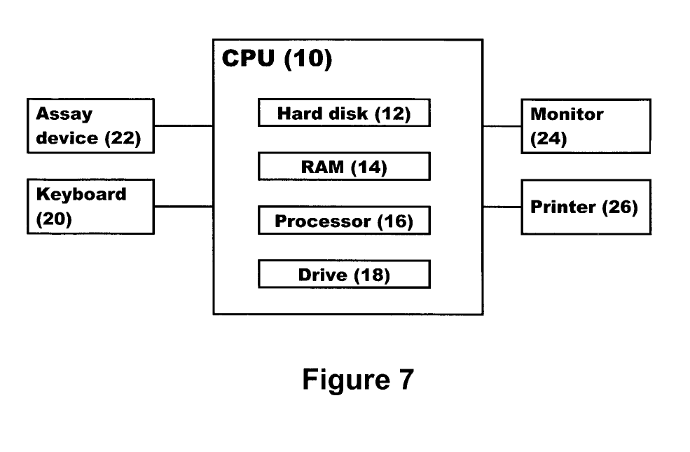

FIG. 4. is a schematic of the human glycolytic pathway and of the polyol pathway, adapted from [22]. Abbreviations additional to those used in FIG. 1 are:

NAD+, nicotinamide adenine dinucleotide;

NADH, nicotinamide adenine dinucleotide, reduced;

NADP, nicotinamide adenine dinucleotide phosphate;

NADPH, nicotinamide adenine dinucleotide phosphate, reduced;

GAPDH, glyceraldehyde-3-phosphate dehydrogenase;

LDH, lactate dehydrogenase;

PDHc, pyruvate dehydrogenase complex;

α-KG, α-ketoglutarate.

FIG. 5 depicts ratios of predicted diabetic over predicted normal myocyte steady state concentrations. Abbreviations additional to those used in FIGS. 1 and 4 are:

GLUe, extracellular glucose;

GLUi, intracellular glucose;

PEP, phosphoenol-pyruvate; PYR, pyruvate;

LAC, lactate; SOR, sorbitol; FRU, fructose.

FIG. 6 depicts ratios of predicted diabetic with aldose reductase inhibition over predicted normal myocyte steady state concentrations. Abbreviations are the same as for FIGS. 1, 4 and 5.

FIG. 7 is a diagram of a computer connected to assay equipment.

### DETAILED DESCRIPTION

The present invention is illustrated by the following description of the construction of a mathematical model of part of the human glycolytic pathway using data for steady-state concentrations in erythrocytes, validation of the model by using data for concentrations in myocytes and application of the model to investigate the glycolytic pathway in another species, T. brucei. The invention is further illustrated by application of an extended model to investigate the glycolytic pathway when diabetes is present.

The human glycolytic pathway is a complex regulated metabolic system, with multiple points of positive and negative feedback. As indicated in FIG. 1a, the glycolytic enzymes convert intracellular glucose into pyruvate. Under anaerobic conditions, pyruvate is subsequently converted to lactate while in aerobic conditions pyruvate enters the Krebs cycle.

For constructing a model, as described here, the first reaction, GLU to G6P was omitted as reliable measured data for the intracellular glucose concentration is not available [14, 17]. In addition, the conversion of FBP to G3P and DHAP was treated as a ‘lumped reaction’ [18]. Hence, the equation describing the FBP/G3P transition also includes the relationships for the transition of FBP/DHAP and DHAP/G3P.

The selection of mathematical expressions to use in the model is illustrated by the following.

Consider a simple single step reaction, in which one mole of substrate A is converted to one mole of product B by an unchanging concentration of the enzyme E1 in a simple single step enzymatic reaction with no allosteric or feedback/forward regulation.

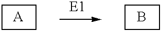

If the substrate A is not replenished from any source, then as the initial one mole quantity of substrate A becomes depleted, the concentration of A (S_{A}), as a function of time t can be represented as:

S_{A}=e^{-k}^{t} (1)

where k is the parameter that determines the rate of reaction. The rate of depletion of A is obtained by differentiating equation (1) with respect to time

$$\frac{ⅆ {S}_{A}}{ⅆ t} = {S}_{A}^{′} = - k {ⅇ}^{- kt} \qquad ( 2 )$$

Because the starting concentration of substrate A was 1 mole, the concentration of B (S_{B}), can be represented as:

S_{B}=1−S_{A} (3)

Combining equations (1) and (3) then leads to the following relationship for S_{B},

S_{B}=1−e^{-kt} (4)

Equation 4 represents an “ideal” substrate/product relationship, in which eventual total conversion of product into substrate is assumed. However, in vivo, physical constraints exist that prevent such a reaction from achieving total completion. For example, in the context of an intact cellular environment, it would be unlikely that substrate A will ever be entirely depleted and conditions must be regarded as non-ideal. Therefore, to match the idealised system (represented in (4)) to its in vivo and non-ideal counterpart, fitting parameters are utilised, as represented in equation (5).

S_{B}=k_{f}(1−e^{-kt}) (5)

where k_{f} is the fitting parameter. In this example, k_{f} is positive and has a value less than 1, i.e. 0<k_{f}<1.

The rate of formation of B can then be obtained by differentiating equation (5) with respect to time

$$\frac{ⅆ {S}_{B}}{ⅆ t} = {S}_{B}^{′} = {k}_{f} k {ⅇ}^{- kt} \qquad ( 6 )$$

The preceding equation, used to dynamically model a single step reaction, can be extended to multiple-step reactions. To illustrate this, consider the situation in which product B is itself a substrate for the production of substance C, and that this reaction is catalysed by enzyme E2, again without any allosteric or feedback/forward regulation.

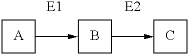

The concentration of product B is dependent on both formation from substrate A and conversion into substance C.

In such a case, equation (5) which gives the concentration SB can be reformulated as equation (7) below. This includes a function (e^{-k2t}) which resembles the function of equation (1) and provides for the depletion of B to C. The function includes an assumed parameter k₂.

S_{B}=k_{f}(1−e^{-kt})e^{-k2t} (7)

The rate of change of B can be expressed by differentiating equation (7) with respect to time:

$$\frac{ⅆ {S}_{B}}{ⅆ t} = {S}_{B}^{′} = {k}_{f} [ k {ⅇ}^{- kt} - {k}_{2} ( 1 - {ⅇ}^{- kt} ) ] {ⅇ}^{- {k}_{2} t} \qquad ( 8 )$$

This principle of putting expressions together can be extended to the entire pathway of interest.

Thus far, this discussion has only considered simple reaction pathways without complex regulatory behaviours such as oscillatory behaviour, or feedback/feedforward inhibition/activation mechanisms. Furthermore, in the above equation (7), B begins from a concentration of zero. This is unlikely to be the case in vivo, as most metabolite concentrations (e.g. glucose) fluctuate around a homeostatic physiologic constant concentration.

These various contributions to the concentration of a metabolite can be described by equations. Thus, oscillatory behaviour is described by an equation of the form:

S_{g1}=k₁sinωt (9)

S_{g1} denotes a contribution to concentration due to oscillatory behaviour, where k₁ can be termed an oscillation coefficient.

The formation of metabolite from its precursor as described by equation (5) above can be rewritten as

S_{g2}=k₂(1−e^{-at} (10)

where k₂ and a are coefficients.

The depletion of metabolite by onward reaction is described by rewriting equation (1) with the form

S_{g3}=k₃e^{-b(t-t}^{₀}⁾ (11)

where t represents the total time course, as in preceding equations, while t₀ represents a time delay before subsequent depletion reaction(s) become significant, and k₃ is a coefficient.

The homeostatic physiological constant concentration is a fixed value which may be written as k_{ST}.

In these equations all constants and parameters are taken as positive.

These equations can be combined and rewritten as a composite function describing concentration of a metabolite:

S_{g}=k₁sinωt+k₂(1−e^{-at})+k₃e^{-b(t31 t0)}+k_{ST} (13)

It is here assumed that formation of metabolite by reversion of the next metabolite in the biochemical pathway is not significant.

If a reaction is reversible, the reverse reaction can be described by means of an equation similar to (10).

S_{gr}=kᵣ(1−e^{-dt}) (10a)

where kᵣ and d are co-efficients.

The next step is that the various parameters in the equation (e.g. k, a, b) are adjusted by an empirical fitting procedure so as to match the model to experimental results. Note, however, that although equation (13), represents a plurality of possible kinetic variables that can take place in a particular reaction, not all expressions in equation (13) are necessary for all reactions. Thus, if oscillatory behaviour is believed to be absent in a particular reaction, S_{g1} is then set to zero.

As an illustration, consider the second reaction of the glycolytic pathway, in which glucose-6-phosphate (G6P) is converted to fructose-6-phosphate (F6P) by phosphoglucoisomerase (PGI). At present, it is believed that in erythrocytes, both metabolite concentrations do not oscillate with time, and that enzymatic co-factors are not required. In this situation, equation (13) reduces to:

S_{g}=k₂(1−e^{-at})+k₃e^{-bt}+k_{ST} (14)

By applying the convolution theory equation (14) can be reduced to:

S_{gB}=k_{2′}(1−e^{-at})e^{-bt}+k_{ST} (15)

where k₂ has been replaced by k_{2′} (i.e. k₂ prime) and addition of two terms in equation (14) has changed to a multiplication as in equation (7).

For complex reactions such as allosteric reactions, some of the parameters can themselves become functions that reflect the feedback or feedforward mechanism being modelled. For example, in the case of the reaction of phosphoenolpyruvate being converted to pyruvate, it is known that increasing the concentration of fructose-1,6-bisphosphate activates the reaction [16], causing an increase in the rate of pyruvate production. Using equation (15) k_{2′} can then be made a function of fructose-1,6-bisphosphate to perform this task. If it is necessary to express an increased rate of reaction (which is obtained by the differentiation of (15)), k_{2′} can then be modified. If the total steady-state concentration is increased as a consequence, k₄ is altered. Such a strategy also holds for reactions in which allosteric inhibition takes place.

S_{gX}=k_{2′}_{ₓ}(S_{gB})(1−e^{-a}^{ₓ}^{t})e^{-b}^{ₓ}^{t}+k_{ST}_{ₓ} (16)

Equation (16) represents the concentration of a metabolite X which derives from a metabolite B but is far downstream in the pathway.

The equation (16) illustrates how an equation can be written to describe concentration when increasing S_{gB} increases the concentration of metabolite X far downstream of a pathway, the (feedforward mechanism).

By combining mathematical expressions in the ways described above, it is possible to establish links between the sequence of reactions in a specified pathway. The form of each expression may be chosen to take into account knowledge of enzyme action and biochemical regulation, but the coefficients used in the expressions are arrived at by empirical fitting to match calculated values with measured values.

In this way the inventors established a set of equations linking the concentrations of the metabolites in the glycolytic pathway. These equations did not require the pathway to be in a steady state.

However, since most of the published experimental literature describes glycolytic data obtained from systems existing at steady state levels, the initial set of equations were transformed to reflect the metabolic behaviour of a cell at steady state. This is illustrated by the following.

At steady state, when time approaches infinity, equation (15) which gives the concentration of an intermediate metabolite becomes

Sg=k_{ST} (17)

As an illustration, consider the first step of the model, in which G6P is converted to FBP. For human erythrocytes, at steady state, when time t is ‘large’, the concentration of G6P approaches a fixed value represented in equation 15 by k_{ST}. In human erythocytes this is 0.039 mM. Equation (15) will therefore become

S_{G6P}=k_{2′}(1−e^{-at})e^{-bt}+0.039 mM (18)

where k_{ST} has been replaced by the actual measured value 0.039 mM. When time t is “large” the first term will approach zero.

Likewise, the equation for the concentration of F6P is taken as

S_{F6P}=k_{3′}(1−e^{-bt})e^{-ct}+k_{4′} (19)

where (1−e^{-bt}) is a term representing formation of F6P from G6P, e^{-ct} is a term representing depletion by the reaction of F6P to FBP, and k₄′ represents steady state concentration. As the conversion of F6B to FBP is an irreversible step, there is no need for any term to express reversion of FBP to F6P. When t is large, F6P concentration approaches k_{4′}.

We can assume that the concentration of a substrate at steady state has a direct impact on the steady-state concentration of the product. Hence, we replace k_{4′} by k_{4″}S_{G6P}, where term k_{4″} is the steady-state coefficient.

S_{F6P}=k_{3′}(1−e^{-bt})e^{ct}+k_{4″}S_{G6P} (20)

By proceeding in this way, modifying the equations on the basis that time t is large, the equations take a form which links the concentrations of the metabolites under steady state conditions. This set of equations constitutes a generic model for the glycolytic pathway. After deciding on the form of each equation in the set, the next stage as mentioned above was to adjust the various coefficients in the equations, so as to get a match between values given by calculation using the equations and measured values of steady state concentrations for human erythrocytes.

Doing this created a steady state erythrocyte model of glycolysis by empirically fitting the generic model to the erythrocyte ‘glycolytic phenotype’ which refers to the concentrations of the various glycolytic metabolites (from glycose-6-phosphate to pyruvate) in this cell type.

Development of the model in this way was performed using a Windows-based GNU Fortran compiler that runs on Windows 95/98 and Windows NT/2000 consoles, and the obtained results were processed using typical spreadsheet applications.

For human erythocytes, the steady-state concentrations (actual values as determined by assay) are known, and are reproduced in a column of Table 1 below.

Fitting the generic model to this erythrocyte data in Table 1, resulted in the definition of a set of steady-state coefficients that generated an optimised model which may be referred to as HEM (human erythrocytes model) or more preferably as HEGM (denoting “human erythrocyte glycolysis model”). The relationships and coefficients incorporated in them were as follows:

| | |
|---|---|
| 1. S_{G6P} = | Input |
| 2. S_{F6P} = | 0.330 × S_{G6P} |
| 3. S_{FBP} = | 0.208 × S_{F6P} |
| 4. S_{G3P} = | 0.660 × S_{FBP}^{[(1.85*S}_{G6P}^{)+0.73]} |
| 5. S_{BPG} = | 0.122 × S_{G3P} |
| 6. S_{3PG} = | 8.000 × S_{BPG}^{0.654} |
| 7. S_{2PG} = | 0.147 × S_{3PG} |
| 8. S_{PEP} = | 1.700 × S_{2PG} |
| 9. S_{PYR} = | 4.950 × S_{PEP} |

Actual concentration values and also predicted values calculated using the HEGM, starting from an initial value which was a concentration of G6P of 0.039 mM, are shown in the following Table 1, and shown graphically in FIG. 1b. The high degree of compliance between the actual and predicted values is revealed by the ratios of the two results in the right hand column of Table 1 (column A/B).

TABLE 1
Measured [19] and predicted glycolytic metabolite
concentrations for erythrocytes

| | A | B | A/B |
|---|---|---|---|
| Metabolites | Measured (mM) | Predicted (mM) | (−) |
| G6P | 0.039 | 0.0390 | 1.00 |
| F6P | 0.013 | 0.0129 | 1.01 |
| FBP | 0.0027 | 0.0027 | 1.00 |
| G3P | 0.0057 | 0.0058 | 0.98 |
| BPG | 0.0007 | 0.0007 | 1.00 |
| 3PG | 0.069 | 0.0705 | 0.98 |
| 2PG | 0.01 | 0.0106 | 0.94 |
| PEP | 0.017 | 0.0180 | 0.94 |
| PYR | 0.085 | 0.0881 | 0.96 |

Two points emerge from this process. Firstly, the empirical fitting performed to create the HEGM does not rely upon changing the underlying mathematical structure of the equations described in the generic model, but on adjusting the numerical value of a restricted subset of parameters. This contrasts with modelling approaches employing traditional enzymatic kinetics, in which additional factors often need to be introduced as the model increases in mathematical complexity [9]. Secondly, the optimised HEGM is fitted to the erythrocyte glycolytic phenotype with high accuracy (FIG. 1b). Again, this contrasts with traditional modelling approaches, in which results within a log scale of variance are often tolerated (i.e. 10-fold or 1000% inaccuracy).

When using a single source of data, it is impossible to determine with confidence the generality of the coefficients used to optimise the model. As such, fitting the model to just one test case is not sufficient to prove its validity. To validate the HEGM, the inventors chose to apply the model to an independent data set of glycolytic measurements, and to compare how well the predicted values calculated from one of these measured values match the remaining measured values of this data set. For this purpose, they chose to use a metabolite concentration series obtained from myocytes. Measured glycolytic metabolite concentrations for myocytes are set out in table 2 below, alongside the values for erthrocytes. Values for myocyte G6P to 2PG were taken from [17] while the PEP [16] was obtained from published Michaelis-Menten curves and PYR from [20]. For myocytes, the BPG value was not available. The ratio of the metabolite concentrations between the myocytes and the erythrocytes is shown in the right hand column (M/E).

TABLE 2
Measured glycolytic metabolite concentrations for
erythrocytes and myocytes

| | Erythrocytes (E) | Myocytes (M) | M/E |
|---|---|---|---|
| Metabolites | (mM) | (mM) | (−) |
| G6P | 0.039 | 0.45 | 11.54 |
| F6P | 0.013 | 0.11 | 8.46 |
| FBP | 0.0027 | 0.032 | 11.85 |
| G3P | 0.0057 | 0.003 | 0.53 |
| BPG | 0.0007 | — | — |
| 3PG | 0.069 | 0.06 | 0.87 |
| 2PG | 0.01 | 0.007 | 0.70 |
| PEP | 0.017 | 0.052 | 3.06 |
| PYR | 0.085 | 0.09 | 1.06 |

Although it is generally believed that myocytes and erythrocytes have grossly similar glycolytic pathways, the specific glycolytic phenotypes of both cell-types are considerably different. For example, the absolute concentrations of metabolites, as well as the ratios between similar metabolites vary across these two cell types in a non-trivial manner as is apparent from Table 2 above. Reasons for these differences may include variations in the expression levels of the various glycolytic enzymes between the cell types, subtle differences in the kinetic and regulatory properties of tissue-specific enzyme isoforms, or the fact that the substrate of glycolysis in myocytes is glycogen rather than glucose [11]. The inventors instructed the HEGM to adopt a starting glucose-6-phosphate concentration of 0.45 mM as found in myocytes and calculate predicted values of the concentrations of the downstream metabolites. Strikingly, they observed that the HEGM, despite being based on an erythrocyte system, was nevertheless capable of generating a series of predicted metabolite concentrations very similar to the actual concentrations found in myocytes (FIG. 2). Specifically, the trends between the predicted to measured metabolite values were fairly similar, and in addition, the accuracy of the predicted metabolite concentrations were within a level generally accepted by biochemists. The maximum deviation was observed at PEP, which exhibits a measured/predicted value of 4.4×. Note that at this point of maximum deviation the measured data is obtained from a different published source (as mentioned above in description of Table 2).

The ability of the unaltered HEGM to predict the glycolytic phenotype for a completely different cell type indicates that the predictive capacity of the HEGM is not confined to the cell type from which it was generated. Indeed, it may be general enough to be used as a model for steady-state glycolysis in numerous vertebrate cell types.

Application in Another Species

It is known that metabolism can vary greatly between different species and different cell-types [12]. As such, identifying the location and nature of these tissue and species-specific differences will be crucial in understanding the mechanisms that underlie biological and phenotypic diversity. The HEGM was used to investigate the glycolytic pathway of Trypanosoma brucei, a parasite that causes African sleeping sickness. The following Table 3 shows that the glycolytic phenotype of T. brucei greatly differs from either erythrocytes or myocytes. Molecularly, numerous differences between the T. brucei and vertebrate glycolytic pathways have also been identified (FIG. 3a) [13, 14].

TABLE 3
Measured glycolytic metabolite concentrations for
erythrocytes [19], myocytes, T. brucei (aerobic) [13] and T. brucei
(anaerobic).[13]

| Metabo- | Erythrocytes | Myocytes | T. brucei | |
|---|---|---|---|---|
| lites | (mM) | (mM) | aerobic (mM) | anaerobic (mM) |
| G6P | 0.039 | 0.45 | 1.64 | 0.74 |
| F6P | 0.013 | 0.11 | 0.9 | 0.61 |
| FBP | 0.0027 | 0.032 | 0.72 | 0.26 |
| G3P | 0.0057 | 0.003 | 0.17 | — |
| BPG | 0.0007 | — | 0.28 | 0.28 |
| 3PG | 0.069 | 0.06 | 4.8 | 1.16 |
| 2PG | 0.01 | 0.007 | 0.59 | 0.33 |
| PEP | 0.017 | 0.052 | 0.74 | 0.39 |
| PYR | 0.085 | 0.09 | 21 | |

For example, T. brucei does not possess a functional Krebs cycle, and thus solely relies on glycolysis for energy production. In addition, in trypanosomes 90% of the glycolytic enzymes are found to be concentrated within an intracellular organelle called the glycosome. Finally, as shown in FIG. 3a, since trypanosomes lack lactate dehydrogenase, the NADH generated during glycolysis is reoxidised by molecular oxygen via a dihydroxyacetone phosphate (DHAP): glycerol-3-phosphate (Gy3P) shuttle in combination with a terminal Gy3P oxidase in the mitochondrion.

As can be seen from the measured values quoted in Table 3, the metabolite concentrations observed in T. brucei are very different from those in human erythrocytes and myocytes.

Although these molecular differences are known, it is unclear which of these differences, in vivo, is functionally responsible for causing the differences between the trypanosomal and vertebrate glycolytic phenotypes.

The inventors first instructed the HEGM to adopt an initial glucose-6-phosphate concentration of 1.64 mM as found in trypanosomes, and calculate predicted concentrations of downstream metabolites. As seen in FIG. 3b, unlike myocytes, the HEGM consistently under-predicts all steady-state concentrations. A closer inspection of the predicted results, however, reveals that the failure of the HEGM to predict the metabolite concentrations of T. brucei is actually biphasic. Specifically, for the first 2 steps of the pathway (from G6P to FBP), the deviation between the experimental and predicted values are between 1-6 fold, which is still within acceptable error ranges, considering that an unoptimised model is being used. However, from G3P onwards, the experimental to predicted values differ much more drastically (>500 fold), and for these downstream steps, the HEGM fails to exhibit any significant predictive power.

This would be evidence (confirmation in the present instance) that the glycolytic pathway of T. brucei is different from that of human erythrocytes and myocytes.

As mentioned above, it is known that a trypanosomal-specific molecular shuttle acts at this step (G3P via DHAP) (FIG. 3a). The inventors hypothesised that the bifurcation between the experimental and predicted metabolite concentrations after G3P might be due primarily to the existence of this trypanosomal-specific metabolic input, which is not modelled by the HEGM (as is based upon vertebrate glycolysis). To test this hypothesis, the inventors first determined if alterations at a single discrete location in the pathway could explain most of the observed differences between the glycolytic phenotypes of vertebrates and trypanosomes. An iterative analysis was performed in which the naive HEGM was instructed to adopt successfully metabolite concentrations matching those found in trypanosomes at various points in the pathway and in each case calculate a set of predicted concentrations of the downstream metabolites. The ability of the HEGM to model subsequent downstream steps was thus observed. The inventors found that instructing the HEGM to adopt matching metabolite concentrations for F6P, FBP and G3P failed to improve the predictive accuracy of the model. FIG. 3c shows the predicted concentration of metabolites downstream of G3P after adopting the measured value of 0.17 mM as the concentration of G3P.

After instructing the HEGM to adopt the measured concentration of 0.28 mM for BGP, the next metabolite, there was a striking and qualitative improvement in the ability of the HEGM to model the remainder of the pathway (FIG. 3d). This result implies that after BPG, the steady-state coefficients used in the HEGM, despite being based upon a vertebrate glycolytic system, can nevertheless be used to model the analogous steps in trypanosomes, and that the glycolytic pathways at these steps may be grossly similar in both species.

Taken collectively, these results indicate that the glycolytic pathways of vertebrates and trypanosomes behave similarly both upstream and downstream of G3P, and that differences between the two systems in their treatment of G3P is sufficient to largely explain the differences in their observed glycolytic phenotypes.

One prediction from this hypothesis is then that altering the HEGM at this single crucial step, but leaving the other steps unchanged, should nevertheless give rise to a model that more accurately reflects the trypanosomal glycolytic phenotype. Alternatively, if the differences between the trypanosomal and vertebrate glycolytic phenotypes are due to factors which operate at multiple points in the pathway, such as differences in enzyme concentrations, regulatory capacities or compartmentalisation of multiple glycolytic enzymes in the glycosome, then one would expect that changing the HEGM at one single step would not yield a model of improved accuracy.

To test this possibility, the inventors altered the HEGM by modifying the steady-state coefficient associated with the G3P to BPG transition from 0.122 to 1600 and applied the new model (referred to as HEGM^{tr}) to the T. brucei metabolic phenotype. As seen in FIG. 3e, HEGM^{tr}, which consists of the initial HEGM with a single altered step, exhibited a striking improvement in its ability to predict the entire T. brucei metabolic phenotype. These results suggest that despite the many molecular differences between the T. brucei and vertebrate glycolytic pathways (described above), the activity of the trypanosomal-specific DHAP:Gy3P molecular shuttle is the key functional network responsible for the differences in the expression of the vertebrate and trypanosomal steady-state glycolytic phenotypes. Such an insight into the consequences of types of differences between cellular pathways would have been difficult to achieve without the availability of a sufficiently accurate and generally applicable computational model. It is currently impractical to address such issues through traditional experimental means.

The following description illustrates the application of the model as a discovery tool to identify, a priori, the locations of functionally important differences between two metabolic pathways. The two pathways considered are provided by the same cell type in distinct physiologic states.

T. brucei, like most organisms, can generate ATP under aerobic or anaerobic condition [13]. However, the specific glycolytic phenotype of T. brucei is also highly dependent upon metabolic state (aerobic vs. anaerobic). The absolute concentrations of glycolytic metabolites of T. brucei under aerobic conditions differ considerably from the same metabolites under anaerobic conditions. The maximum deviations are observed at 3PG and Gy3P (as shown in Table 3 above).

A set of equations was selected to provide a model for T. brucei glycolysis under aerobic conditions. This model was referred to as the TBAE model. As compared to the HEGMtr model, the equation coefficients were optimised for T. brucei aerobic glycolysis and the model included additional equations for the branch metabolites DHAP and Gy3P.

After confirming that this new model could accurately predict the glycolytic phenotype of T. brucei under aerobic conditions, the inventors instructed the model to adopt an initial G6P value of 0.74 mM as found in T. brucei under anaerobic conditions. Despite the considerable differences in glycolytic phenotypes in aerobic and anaerobic T. brucei, the aerobic model was able to accurately predict, to within a factor of 2×, the anaerobic concentrations of all but one of the metabolites downstream of G6P (FIG. 3f). The sole metabolite whose concentration was not accurately predicted, however, was Gy3P, which was under-predicted by a factor of 8.9×. Notably, metabolites in the core glycolytic pathway such as BPG to PEP, which occupy positions further “downstream” than Gy3P, were all still accurately predicted.

This indicates a functional difference between the glycolytic networks of aerobic and anaerobic T. brucei at this point (Gy3P). A search of the available literature subsequently revealed that under anaerobic conditions, a state transition occurs in which Gy3P now becomes converted to glycerol via the activity of glycerol kinase. This pathway, which is inactive [14] or minimally active [21] under aerobic conditions, exhibits dramatic activity in an anerobic setting.

This finding was obtained using the same model for two states of a single organism.

Another version of the HEGM was developed using equations of the general form

S_{G}=k₁(1−e^{-k}^{₂}^{t})(e^{-k}^{₃}^{t})+k4S_{(G-1)}^{k}^{₅}

where S_{G} denotes the concentration of metabolite and S_{(G-1)} denotes the concentration of the preceding metabolite.

Time t was taken as 60 seconds, taken as a time by which the reactions would have proceeded some way, but without reaching the steady state.

The parameters k₁, k₂, k₃, k₄, k₅ were adjusted to fit the equations to known data for the metabolites of the erythrocyte glycolytic pathway as given in Table 4 below. The values of the coefficients after fitting were as follows:

TABLE 4
Values of coefficients in equations

| Metabolite | k₁ | k₂ | k₃ | k₄ | k₅ |
|---|---|---|---|---|---|
| F6P | 0.22 | 0.02 | 0.09 | 0.25 | 1.0 |
| FBP | 0.1 | 0.09 | 0.19 | 0.25 | 1.0 |
| G3P | 0.35 | 0.19 | 0.29 | 0.6 | 1.85 * S_{G6P} + 0.73 |
| BPG | 0.05 | 0.1 | 0.2 | 0.122 | 1.0 |
| 3PG | 0.04 | 0.2 | 0.01 | 6.9 | 0.7 |
| 2PG | 0.5 | 0.01 | 0.1 | 0.11 | 1.0 |
| PEP | 0.005 | 0.1 | 0.07 | 1.6 | 1.0 |
| PYR | 0.07 | 0.07 | 0.01 | 1.5 | 1.0 |

When this version of the model was used to calculate predicted values to be compared with measured values (steady state concentrations)it gave similar predictions to those discussed above.

The robustness or sensitivity of this version of the model was investigated by modifying the coefficients used in the equations. When the coefficients were increased by 5%, and also when they were decreased by 5%, the model continued to give the results described above. However, the model failed completely when all coefficients were arbitrarily set at 1.0.

In summary, the results indicate that although most cell types behave as dynamic entities, modelling a cell's steady-state behaviour in terms of metabolite relationships can be useful in elucidating important cellular functions. Using glycolysis as an example, the inventors have shown that a complex metabolic pathway can be accurately modelled using an empirical strategy, and that, once fitted, the model is general enough to be applied to different systems. In addition, without prior knowledge of the molecular differences between systems, this strategy is also capable of rapidly identifying the locations of functionally important differences in metabolic pathways. Recent reports have highlighted the use of metabolite concentrations to investigate unexplored areas such as ‘silent’ gene functions [15]. However, experimental efforts in this area have typically proved expensive and laborious. The availability of a computational model as provided by the present invention should benefit researchers by allowing them to perform such analyses in silico, and consequently at greatly reduced time and cost. In addition, such approaches will also ultimately prove useful in more translational areas such as drug design, as the targeting of tissue and species-specific activities may be one promising strategy to reduce the pleiotropic side-effects associated with conventional medications, and yet offer an avenue through which common physiologic pathways can still be perturbed.

The investigations described above demonstrate that a set of mathematical expressions developed as a relationship between metabolites in a known biochemical pathway can be applied to a different biochemical pathway in which the same metabolites occur. Comparison is made between measured values for concentrations of metabolites in this different biochemical pathway and predicted values for the same metabolites calculated using only one of the measured values. This reveals that the second biochemical pathway differs from the known pathway. Further investigation which also makes a comparison between measured and predicted values for the different biochemical pathway elucidates where the difference between that pathway and the known pathway lies.

In this work described above, for the purposes of demonstration, the different biochemical pathway was one which has already been described in the literature.

However the same procedure could be employed for investigating other pathways. For instance the HEGM could be employed with measured values obtained by assay of a sample of human material which had been exposed to the action of a drug or potential drug, and thereby used to investigate whether that drug or potential drug was acting within the glycolytic pathway.

Models for other biochemical pathways can be developed following the same principles as explained above and likewise used for investigating the site of action of drugs or potential drugs.

Altered Metabolism in Diabetes

The glycolysis model was further extended for the study of myocardial metabolic alteration that occurs in diabetic patients.

Under conditions of elevated blood glucose, which is characteristic of diabetes, there is increased glucose metabolism via the polyol pathway in which glucose is reduced to sorbitol by aldose reductase in the presence of NADPH, and sorbitol is then oxidized by sorbitol dehydrogenase to fructose at the cost of NAD+, see FIG. 4. This has been demonstrated to result in impaired utilization of glycolytic substrates in diabetic rat hearts [22].

It has been proposed that the inhibition of aldose reductase would make the polyol pathway ineffective and thus tend to restore glucose metabolism via the normal glycolysis pathway.

A set of equations to model this system was devised. First the parameters of the HEGM model described above were adjusted to reflect the measured data of myocytes [17].

The resulting model, termed MGM (myocyte glycolysis model) was then extended to include the polyol pathway, glucose transport, lactate production and the Krebs cycle. This extended model is referred to as MEM (myocyte extended model). Extending the model also involved some modifications to the equations which were used. Notably, equations relating to enzymatic reactions which involve NAD⁺ as a co-factor were modified to include a term S_{NAD} denoting the concentration of NAD⁺.

The fifteen equations used in MEM are listed below and values of the coefficients in these equations optimized for myocyte data are listed in the following Table. These equations include time dependent terms, and time was set at 60 seconds, being a time by which reactions would have proceeded some way.

For extra cellular glucose concentration:

S_{GLUe}=k₁(k₂e^{-k}^{₃}^{t}+k₄S_{GLUi}+k₅) (MEM1)

For intracellular glucose concentration:

$${S}_{GLUi} = {k}_{1} ( 1 - {ⅇ}^{- {k}_{2} t} ) {ⅇ}^{- {k}_{3} t} + \frac{{k}_{4}}{{S}_{GLUe}} \qquad ( MEM2 )$$

For sorbitol concentration:

$${S}_{SOR} = ( \frac{{k}_{1}}{{S}_{GLUi}} ) ( 1 - {ⅇ}^{- {k}_{2} t} ) {ⅇ}^{- {k}_{3} {S}_{{NAD}^{t}}} + {k}_{4} \qquad ( MEM3 )$$

For fructose concentration:

S_{FRU}=k₁(1−e^{-k}^{₂}^{t})+k₃S_{NAD}+k₄S_{SOR} (MEM4)

For glucose-6-phosphate concentration:

$${S}_{G6P} = {k}_{1} ( 1 - {ⅇ}^{- {k}_{2} t} ) {ⅇ}^{- {k}_{3} t} + \frac{{k}_{4} {S}_{GLUi}}{{S}_{SOR}} \qquad ( MEM5 )$$

For fructose-6-phosphate concentration:

S_{F6P}=k₁(1−e^{-k}^{₂}^{t})e^{-k}^{₃}^{t}+k₄S_{G6P} (MEM6)

For fructose-1,6-bisphosphate concentration:

S_{FBP}=k₁(1−e^{k}^{₂}^{t})e^{-k}^{₃}^{t}+k₄S_{F6P} (MEM7)

For glyceraldehyde3-phosphate concentration:

$${S}_{G3P} = {k}_{1} ( 1 - {ⅇ}^{- {k}_{2} t} ) {ⅇ}^{- {k}_{3} t} + {k}_{4} {S}_{FBP} + \frac{{k}_{5}}{{S}_{NAD}} \qquad ( MEM8 )$$

For 1,3-bisphosphoglycerate concentration:

S_{BPG}=k₁(1−e^{k}^{₂}^{t})e^{-k}^{₃}^{t}+k₄S_{G3P}+k₅S_{NAD} (MEM9)

For 3-phosphoglycerate concentration:

S_{3PG}=k₁(1−e^{-k}^{₂}^{t})e^{-k}^{₃}^{t}+k₄S_{BPG} (MEM10)

For 2-phosphoglycerate concentration:

S_{2PG}=k₁(1−e^{-k}^{₂}^{t})e^{-k}^{₃}^{t}+k₄S_{3PG} (MEM11)

For phosphoenol-pyruvate concentration:

S_{PEP}=k₁(1−e^{k}^{₂}^{t})e^{-k}^{₃}^{t}+k₄S_{2PG} (MEM12)

For pyruvate concentration:

S_{PYR}=k₁(1−e^{k2t})e^{-k3t}+k4S_{PEP}+k5S_{NAD} (MEM13)

For lactate concentration:

$${S}_{LAC} = {k}_{1} ( 1 - {ⅇ}^{- {k}_{2} t} ) + {k}_{3} {S}_{PYR} + \frac{{k}_{4}}{{S}_{NAD}} \qquad ( MEM14 )$$

For nicotinamide adenine dinucleotide concentration:

$${S}_{NAD} = {k}_{1} ( {k}_{2} + {k}_{3} sin {k}_{4} t ) + \frac{{k}_{5}}{{S}_{SOR}} \qquad ( MEM15 )$$

TABLE 5
Coefficients in MEM equations 1 to 15 above

| | Metabolites | k₁ | k₂ | k₃ | k₄ | k₅ |
|---|---|---|---|---|---|---|
| MEM1 | S_{GLUe} | k | 5.25 | 0.009 | −0.01 | 5 |
| MEM2 | S_{GLUi} | 10 | 0.1 | 1 | 7.5 | — |
| MEM3 | S_{SOR} | 0.5 | kk | 0.003 | 0.1 | — |
| MEM4 | S_{FRU} | 0.4 | 0.003 | 0.1 | 0.1 | — |
| MEM5 | S_{G6P} | 0.22 | 0.3 | 0.02 | 0.18 | — |
| MEM6 | S_{F6P} | 0.2 | 0.02 | 0.04 | 0.2 | — |
| MEM7 | S_{FBP} | 0.18 | 0.04 | 0.19 | 0.29 | — |
| MEM8 | S_{G3P} | 0.162 | 0.19 | 0.1 | 0.0004 | 0.00007 |
| MEM9 | S_{BPG} | 0.05 | 0.1 | 0.2 | 0.001 | 0.012 |
| MEM10 | S_{3PG} | 0.05 | 0.2 | 0.01 | 70 | — |
| MEM11 | S_{2PG} | 0.5 | 0.01 | 0.1 | 0.12 | — |
| MEM12 | S_{PEP} | 0.009 | 0.1 | 0.07 | 7.3 | — |
| MEM13 | S_{PYR} | 0.03 | 0.07 | 0.01 | 1.2 | 0.35 |
| MEM14 | S_{LAC} | 0.07 | 0.01 | 0.2 | 0.6 | — |
| MEM15 | S_{NAD} | 2/k | 0.01 | 0.1 | 0.1 | 0.005 |

Time t = 60 seconds in all equations
k = 1 for normal condition and k = 5 for diabetic condition.
kk = 0.35 as default. With aldose reductase inhibition, kk = 0.01

In deciding the form of the equations and some of their coefficients, empirical choices were included so that by altering values assigned to two coefficients, the model could provide for normal and diabetic conditions and could also reflect inhibition of aldose reductase.

To model normal metabolism, the parameter k (which is coefficient k₁ in the equation for extra cellular glucose concentration) is set as 1 and parameter kk (which is coefficient k₂ in the equation for sorbitol formation by means of the enzyme aldose reductase) was set at 0.35. With these parameters the model gave a good prediction of normal metabolite concentrations.

It will be seen from the equations that after a value has been assigned to the parameter k, the concentrations of extracellular and intracellular glucose S_{GLUe} and S_{GLUi} can be calculated using the first two of the equations. Setting the parameter k to 1 causes the model to calculate a concentration of extracellular glucose which matches an experimantally measured value in normal, non-diabetic metabolism.

To model diabetic conditions the parameter k was raised to 5 reflecting higher extra cellular glucose concentration. The model then generated predicted values under diabetic conditions, including a value for extracellular glucose concentration which matches a representative value for the concentration observed in diabetic patients.

The bar chart which is FIG. 5 shows the predicted metabolite concentrations under diabetic conditions as a ratio of the corresponding predicted concentrations under normal conditions. It can be seen that none of these ratios is equal to one, indicating that all the concentrations change under diabetic conditions. As shown by this bar chart, the model for the diabetic condition predicts that concentrations of G3P, sorbitol (SOR), fructose (FRU) and NAD⁺ would be raised compared with the corresponding metabolite concentrations under normal conditions. This is in accordance with experimental observation [22, 24]. This is an indication of the validity of the model to give a prediction of metabolite concentrations under diabetic conditions.

In order to be able to model the effect of aldose reductase inhibition, the term k4 which is a constant in the equation for sorbitol concentration is given a low value of 0.1. The effect of an aldose reductase inhibitor can be predicted by reducing the parameter kk (which is the coefficient k2 in the equation for the concentration of sorbitol) from 0.35 to 0.01. At the chosen time t=60 seconds this alters the model in a manner consistent with a reduced rate of conversion of glucose into sorbitol.

When the MEM was executed with k=5 and kk=0.01, predicted values of concentration showed trends which matched published experimental data [22]. The bar chart which is FIG. 6, shows the predicted metabolite concentrations under diabetic conditions as a ratio of the corresponding predicted concentrations under normal conditions. It can be seen that that the ratios of the predictions are closer to one for many of the metabolites, notably including glyceraldehyde-3-phosphate, lactate, sorbitol and fructose. However, some of the predicted concentrations still differ from normal values, and in particular the concentration of lactate is still three times the normal concentration.

This is consistent with statements in the literature that aldose reductase inhibition improves glycolysis [22, 24] and yet aldose reductase inhibitors have not been successful during clinical trials [23].

Apparatus

When this invention is put into effect, the calculation and comparison steps will most conveniently be carried out using data processing apparatus. Such apparatus may be a desk top computer running an application program in accordance with this invention as indicated earlier. Input of mathematical expressions or input of a selection from a library of expressions contained within a program may be carried out through a keyboard. Input of measured values of concentration may also be through a keyboard.

Measurement of concentrations may be carried out by typical biochemical assay techniques. If the invention is being used for drug screening so that the reference biochemical pathway is a normal biochemical pathway and the biochemical pathway under investigation is embodied in biological samples which have been exposed to a drug or potential drug, then it may be desirable to use automated or semi-automated analytical apparatus. It is possible that such apparatus will deliver measured values of concentration directly into a computer to which it is connected rather than merely printing out data which must then be input into the computer by hand (although the latter is by no means ruled out).

An embodiment of suitable apparatus is illustrated diagrammatically in FIG. 7 of the drawings in which a desk top computer (10) incorporates non-volatile memory such as a hard disk (12) and volatile RAM (14) as well as processor (16) and a drive (18) for removable data carriers such as a floppy disk drive or a CD ROM drive. This computer is connected to a keyboard (20) for input of data and instructions and to automated assay equipment (22). It is also connected to a conventional monitor (24) capable of displaying a quality measure as numerical or graphical output and is also connected to a printer (26).

### REFERENCES

1. Lander, E. S. et al, Nature 409, 860, (2001). Venter, J. C. et al, Science 291, 1304, (2001).

2. Tan, P. and S. Kim, Trends in Genetics 15, 145, (1999). Simon, M. A., Cell 103, 13, (2000).

3. Alon, U., M. G. Surette, N. Barkai, and S. Leibler, Nature 397, 168, (1999). Barkai, N. and S. Leibler, Nature 387, 913, (1997). Bhalla, U. S. and R. Iyengar, Science 283, 381, (1999). Bray, D., M. Levin, and C. J. Morton-Firth, Nature 393, 85, (1998). Elowitz, M. B. and S. Leibler, Nature 403, 335, (2000). Hartwell, L. H., J. J. Hopfield, S. Leibler, and A. W. Murray, Nature 402, C47-C52, (1999). Palsson, B., Nature Biotechnol. 18, 1147, (2000).

4. Fell, D. A., Understanding the control of metabolism (Portland Press, London, 1997). Tomita, M., et al, Bioinformatics 15, 72, (1999).

5. Schuster, S., T. Dandekar, and D. A. Fell, Trends Biotech. 17, 53, (1999).

6. Bailey, J. E., Nature Biotech. 19, 503, (2001).

7. Varner, J. and D. Ramkrishna, Curr. Opin. Biotechnol. 10, 146, (1999).

8. Ovadi, J. and P. A. Srere, Cell Biochem. Funct. 14, 249, (1996). Rohwer, J. M., P. W. Postma, B. N. Kholodenko, and H. V. Westerhoff, Proc. Natl. Acad. Sci. U.S.A. 95, 10547, (1998).

9. Cornish-Bowden, A., Enzyme kinetics. (In focus, Rickwood, D. IRL Press, 1988).

10. Proc ASHREA Conference 4, 283 (1995).

11. Fell, D. A., Adv Enzyme Regul 40, 35, (2000).

12. Kather, B., S. K., M. E. van der Rest, K. Altendorf, and D. Molenaar., J. Bacteriol. 182, 3204, (2000). Pollack, J. D., M. V. Williams, and R. N. McElhaney, Crit Rev Microbiol 23, 269, (1997).

13. Opperdoes, F. R., et al, Journal of Cell Biology 98, 1178, (1984). Visser, N. and F. R. Opperdoes, Eur. J. Biochem. 103, 623, (1980). Wiemer, E. A. C., P. A. M. Michels, and F. R. Opperdoes, Biochem. J. 312, 479, (1995).

14. Bakker, B. M., P. A. Michels, F. R. Opperdoes, and H. V. Westerhoff, J. Biol. Chem. 272, 3207, (1997).

15. Raamsdonk, L. M., et al, Nature Biotech. 19, 45, (2001).

16. Ikeda, Y. and T. Noguchi, J. Biol. Chem. 273, 12227, (1998).

17. Fersht, A., Enzyme Structure and Mechanism (W H Freeman and Company, ed. 2, 1985).

18. Jana Wolf, et al, Biophysical Journal. 78, 1145, (2000).

19. Peter J. Mulquiney and Philip W. Kuchel, Biochemical Journal. 342, 581, (1999).

20. Proceedings of the Society for Experimental Biology and Medicine 223, 136 (2000).

21. Eisenthal, R. and Cornish-Bowden, A., J. Biol. Chem. 273, 5500, (1998).

22. Trueblood, N. & Ramasamy R., Am. J. Physiol. 275 (Heart Circ. Physiol. 44), H75, (1998).

23. Mendosa, R. Diabetes Wellness Letter, January 1999, pages 1, 3-4.

24. Ramasamy R., Trueblood, N. & Schaefer S., Am. J.

Physiol. 275 (Heart Circ. Physiol. 44), H195, (1998).

1. A computational method of investigating the effect of a test substance on a biochemical pathway comprising the following steps:
storing a set of mathematical expressions which are effective to calculate values representing pre-selected concentrations of metabolites in a pre-selected, reference biochemical pathway from an initial value representing the measured concentration of one metabolite in said reference biochemical pathway;
treating an embodiment of a biochemical pathway with at least one test substance and thereafter assaying at least one test sample of the treated pathway to measure the concentration of at least one metabolite lying within the treated biochemical pathway;
storing values representing said measured concentrations;
using said mathematical expressions to calculate a predicted value of concentration of at least one metabolite in said treated pathway from a value representing the measured concentration of one other metabolite in said pathway;
comparing at least one said predicted value with the corresponding value representing the measured metabolite concentration to obtain a comparison relationship; and
deriving a quality measure of the match between two biochemical pathways on the basis of the comparison relationship.

2. A method according to claim 1 which comprises storing said expressions, said values representing measured concentrations and said predicted values in computer memory.

3. A method according to claim 1 wherein said measured concentrations are concentrations under steady state conditions.

4. A computational method of investigating the effect of a test substance on a biochemical pathway comprising:
storing a set of mathematical expressions which are effective to calculate values representing pre-selected metabolite concentrations in a pre-selected, reference biochemical pathway from an initial value representing the measured concentration of one metabolite in said reference biochemical pathway
storing a set of values representing measured concentrations of the metabolites in a second biochemical pathway containing the same metabolites as the reference pathway;
using said mathematical expressions to calculate a predicted value of concentration of at least one said metabolite in said second biochemical pathway from an initial value representing the measured concentration of one metabolite in said second pathway;
comparing said predicted values with corresponding values representing measured metabolite concentration to obtain a comparison relationship;
modifying at least some of the expressions so as to match predicted values calculated therefrom to the values representing measured metabolite concentrations of the second biochemical pathway, thereby providing a set of expressions modelling said second pathway;
treating a biochemical pathway with at least one test substance to provide a third biochemical pathway containing the same metabolites as the reference and second pathways and thereafter assaying a test sample thereof to measure the concentration of at least one metabolite lying within the third biochemical pathway;
storing values representing said measured concentrations in said third pathway;
using said mathematical expressions to calculate a predicted value of concentration of at least one metabolites in the third pathway from a value representing the measured concentration of one other metabolite in said third pathway;
comparing at least one said predicted value with the corresponding value representing the measured metabolite concentration in said third pathway to obtain a comparison relationship; and
deriving a quality measure of the match between two biochemical pathways on the basis of the comparison relationship.

5. A method according to claim 4 which comprises storing said expressions, said values representing measured concentrations and said predicted values in computer memory.

6. A method according to claim 4 wherein said measured concentrations are concentrations under steady state conditions.

7. A method of creating a computational representation of a biochemical pathway comprising a first metabolite and a plurality of further metabolites whose concentrations are directly or indirectly dependent on the concentration of the first metabolite, by steps of
choosing a set of mathematical expressions for calculating predicted values representing concentrations of said further metabolites from an initial value representing a measured concentration of the first metabolite in an embodiment of the biochemical pathway;
modifying at least some of the expressions so as to match the calculated values to a set of reference values representing measured concentrations in the embodiment of the biochemical pathway; and
storing the expressions in memory.

8. A method according to claim 7 comprising storing the expressions in computer memory.

9. A method according to claim 7 comprising storing the expressions on a computer-readable data carrier.

10. A method according to claim 7 including a step of assaying a plurality of samples of said pathway, to determine said initial and reference values representing measured concentrations.

11. A method according to claim 7 wherein said concentrations are concentrations under steady state conditions.

12. A method according to claim 7 comprising a further step of using said expressions to calculate predicted values representing concentrations of said further metabolites from a value representing the concentration of a metabolite measured in a second embodiment of the biochemical pathway and comparing these predicted values with values representing measured concentrations in said second embodiment of the pathway, thereby to validate the set of expressions as a model for the biochemical pathway.

13. A computational method of investigating a biochemical pathway comprising:
storing a set of values representing measured concentrations of a set of metabolites in a biochemical pathway under investigation;
calculating a predicted value of concentration of at least one metabolite in said pathway from an initial value representing the measured concentration of one other metabolite in said pathway under investigation;
comparing one or more predicted values with corresponding values representing the measured metabolite concentrations to obtain a comparison relationship; and
deriving a quality measure of the match between the biochemical pathway under investigation and another biochemical pathway containing these metabolites on the basis of the comparison relationship.

14. A method according to claim 13 comprising storing said values representing measured concentrations and said predicted values in computer memory.

15. A method according to claim 13 wherein said measured concentrations are concentrations under steady state conditions.

16. A method according to claim 13 which also comprises providing, on a data carrier, a set of mathematical expressions which are effective to calculate values representing metabolite concentrations in a reference biochemical pathway, and said step of calculating predicted values is performed using said mathematical expressions, and the quality measure of match is between the biochemical pathway under investigation and the reference biochemical pathway.

17. A method according to claim 13 which also comprises assaying the concentration of at least one metabolite in at least one test sample of the biochemical pathway under investigation prior to storing values representing measured concentrations.

18. A method according to claim 13 which also comprises treating a biochemical pathway with a test substance to provide the biochemical pathway under investigation.

19. A method according to claim 13 which also comprises
calculating a further predicted value of the concentration of at least one said metabolite in said biochemical pathway under investigation from a value representing the measured concentration of one of the metabolites in said pathway other than the metabolite represented by the said initial value;
then comparing the further predicted values with corresponding values representing measured metabolite concentrations so as to obtain a further comparison relationship; and
deriving a further quality measure of the match between biochemical pathways on the basis of the further comparison relationship.

20. A computational investigative method comprising:
storing a set of mathematical expressions which are effective to calculate values representing pre-selected metabolite concentrations in a pre-selected, reference biochemical pathway from an initial value representing the measured concentration of one metabolite in said reference biochemical pathway;
storing a set of values representing measured concentrations of the metabolites in a second biochemical pathway containing the same metabolites as the reference pathway;
using said mathematical expressions to calculate a predicted value of concentration of at least one said metabolite in said second biochemical pathway from an initial value representing the measured concentration of one metabolite in said second pathway;
comparing one or more said predicted values with corresponding values representing measured metabolite concentrations to obtain a comparison relationship; and
deriving a quality measure of the match between two biochemical pathways on the basis of the comparison relationship.

21. A method according to claim 20 which comprises storing said expressions, said values representing measured concentrations and said predicted values in computer memory.

22. A method according to claim 20 wherein said measured concentrations are concentrations under steady state conditions.

23. A method according to claim 20 which also comprises assaying the concentration of at least one said metabolite in at least one test sample of said second biochemical pathway prior to storing values representing measured concentrations.

24. A method according to claim 20 which also comprises treating a biochemical pathway with a test substance to provide said second biochemical pathway.

25. A method according to claim 20 wherein the quality measure of match is between said second biochemical pathway and the pre-selected, reference biochemical pathway.

26. A method according to claim 20 which also comprises
calculating at least one further predicted value of the concentration of at least one said metabolite in said second biochemical pathway from a value representing the measured concentration of one of the metabolites in said pathway other than the metabolite represented by said initial value;
then comparing the further predicted values with corresponding values representing measured metabolite concentrations so as to obtain a further comparison relationship; and
deriving a further quality measure of the match between biochemical pathways on the basis of the further comparison relationship.

27. A method according to claim 20 which also comprises
storing a set of values representing measured concentrations of metabolites in a third biochemical pathway containing the same metabolites as the reference pathway;
using said mathematical expressions to calculate a predicted value of concentration of at least one said metabolite in said third biochemical pathway from an initial value representing the measured concentration of one metabolite in said third pathway;
comparing one or more said predicted values for said third pathway with corresponding values representing measured metabolite concentrations in said third pathway, so as to obtain a comparison relationship and deriving a quality measure of the match between two biochemical pathways on the basis of the comparison relationships for said third pathway.

28. A method according to claim 27 wherein the quality measure of match is between said third biochemical pathway and the pre-selected, reference biochemical pathway.

29. A method according to claim 27 wherein the quality measure of match is between said second biochemical pathway and said third reference biochemical pathway.

30. A method according to claim 27 which also comprises treating a biochemical pathway with at least one test substance to provide said third biochemical pathway.

31. A method according to claim 27 comprising treating a biochemical pathway with at least one test substance to provide said third biochemical pathway and thereafter assaying at least one test sample thereof to measure concentrations of at least one metabolite lying within said third biochemical pathway.

32. A method according to claim 20 which also comprises modifying at least some of the expressions so as to match predicted values calculated therefrom to the values representing measured metabolite concentrations of the second biochemical pathway, thereby providing a set of expressions modelling said second pathway;
storing a set of values representing measured concentrations of metabolites in a third biochemical pathway containing the same metabolites as the reference and second pathways;
using said mathematical expressions modelling said second pathway to calculate a predicted value of concentration of at least one said metabolite in said third biochemical pathway from an initial value representing the measured concentration of one metabolite in said third pathway;
comparing at least one said predicted value for said third pathway with the corresponding value representing measured metabolite concentration in said third pathway, so as to obtain a comparison relationship for the third pathway and deriving a quality measure of the match between said two biochemical pathways on the basis of the comparison relationships for said third pathway.

33. A method according to claim 32 wherein the quality measure of match is between said third biochemical pathway and the pre-selected, reference biochemical pathway.

34. A computational investigative method for a biochemical pathway comprising a first metabolite and a plurality of further metabolites whose concentrations are directly or indirectly dependent on the concentration of the first metabolite, comprising steps of
choosing a set of mathematical expressions for calculating predicted values representing concentrations of said further metabolites from an initial value representing a measured concentration of the first metabolite in a reference biochemical pathway;
modifying at least some of the expressions so as to match the calculated values to a set of reference values representing measured concentrations in the reference biochemical pathway; and
storing the expressions in memory;
storing a set of values representing measured concentrations of the metabolites in a second biochemical pathway containing the same metabolites as the reference pathway;
using said mathematical expressions to calculate predicted values of concentration of a plurality of said metabolites in the second pathway from an initial value representing the measured concentration of one metabolite in said second pathway;
comparing predicated values with the corresponding values representing measured metabolite concentrations in said second pathway to obtain a comparison relationship; and
deriving a quality measure of the match between said second biochemical pathway and the pre-selected, reference biochemical pathway on the basis of the comparison relationship.

35. A method according to claim 34 which also comprises assaying concentrations of metabolites in a test sample embodying said second biochemical pathway prior to storing values representing those concentrations.

36. A computer memory product having stored thereon at least one digital file, said memory product comprising computer readable memory and said stored digital file or files constituting a program to carry out a method according to claim 20.

37. A computer memory product having stored thereon at least one digital file, said memory product comprising computer readable memory and said stored digital file or files constituting a computer program for investigating a biochemical pathway comprising
(a) code selected from the group consisting of:
code representing a set of mathematical expressions which are effective to calculate values representing pre-selected concentrations of metabolites in a pre-selected reference biochemical pathway from an initial value representing the measured concentration of one metabolite in said reference biochemical pathway;
code effective to receive input of and store mathematical expressions, code effective to receive input and store selection from among a collection of stored expressions, and combinations thereof; plus
b) code adapted to perform the following steps when the program is run on a data processing system:
receive input of and store a set of values representing measured metabolite concentrations in a biochemical pathway;
use said mathematical expressions to calculate a predicted value of concentration of at least one metabolite in a pathway from an initial value representing the measured concentration of one metabolite in said pathway;
compare one or more said predicted values with corresponding values representing the measured metabolite concentrations to obtain a comparison relationship;
derive a quality measure of the match between two biochemical pathways on the basis of the comparison relationship; and
display or output said quality measure.

38. Apparatus for carrying out the method of claim 20, including
an input device for input of values representing concentrations of metabolites;
a processor for utilising a stored program to perform the steps of using said mathematical expressions to calculate predicted values of concentrations from a value representing one measured concentration, comparing one or more said predicted values with corresponding values representing measured concentrations to obtain a comparison relationship and deriving a quality measure or match on the basis of that comparison relationship;
a display device for said quality measure; and
memory for storage of values, said program and said quality measure.

39. Apparatus for carrying out the method of claim 20, including
an assay device for measurement of concentrations of metabolites in a test sample,
a processor for receiving and storing values representing measured concentrations of metabolites, and
for utilising a stored program to perform the steps of using said mathematical expressions to calculate predicted values of measured concentrations from a value representing one measured concentration, comparing one or more said predicted values with corresponding values representing measured concentrations to obtain a comparison relationship and deriving a quality measure or match on the basis of that comparison relationship;
a display device for said quality measure; and
memory for storage of values, said program and said quality measure.

40. A data carrier which is a computer memory product having recorded thereon a mathematical model obtained by the method of claim 7.

41. A data carrier which is a computer memory product having recorded thereon a set of mathematical expressions which are effective to calculate values representing pre-selected metabolite concentrations in a pre-selected, reference biochemical pathway from an initial value representing the measured concentration of one metabolite in said reference biochemical pathway.
